SEQUENCE LISTING (9 sequences)

SEQ ID NO: 1 - DNA, 15 bp, artificial
  Polyadenylation site
tgggatacaacctct

SEQ ID NO: 2 - DNA, 15 bp, artificial
  Polyadenylation site
atactacacataaca

SEQ ID NO: 3 - DNA, 14 bp, artificial
  Polyadenylation site
gggcattaccagaa

SEQ ID NO: 4 - DNA, 11 bp, artificial
  Polyadenylation site
tgggatacaac

SEQ ID NO: 5 - DNA, 13 bp, artificial
  Polyadenylation site
tgggatacaacct

SEQ ID NO: 6 - DNA, 13 bp, artificial
  Polyadenylation site
ttgggtgactgtc

SEQ ID NO: 7 - DNA, 10 bp, artificial
  Polyadenylation site
attaaaatca

SEQ ID NO: 8 - DNA, 10 bp, artificial
  Polyadenylation site
tttaaaatca

SEQ ID NO: 9 - DNA, 10 bp, artificial
  Polyadenylation site
aaattttttg

(19) United States
(12) Patent Application Publication
(10) Pub. No.: US 2023/0073973 A1
(43) Pub. Date: Mar. 9, 2023

(54) DEEP LEARNING BASED SYSTEM AND METHOD FOR PREDICTION OF ALTERNATIVE POLYADENYLATION SITE

(71) Applicant: KING ABDULLAH UNIVERSITY OF SCIENCE AND TECHNOLOGY, Thuwal (SA)

(72) Inventors: Xin GAO, Thuwal (SA); Zhongxiao LI, Thuwal (SA)

(21) Appl. No.: 17/595,343

(22) PCT Filed: Apr. 23, 2020
(86) PCT No.: PCT/IB2020/053867

§ 371 Date: Nov. 15, 2021

Related U.S. Application Data
(60) Provisional application No. 62/851,898, filed on May 23, 2019.

(51) Int. Cl. G06N 3/12; G06N 3/04; G06N 3/063; G16B 40/20; G16B 20/30

(52) U.S. Cl. CPC G06N 3/126; G06N 3/0445; G06N 3/0454; G06N 3/063; G16B 40/20; G16B 20/30

(57) ABSTRACT

A method for calculating usage of all alternative polyadenylation sites (PAS) in a genomic sequence includes receiving plural genomic sub-sequences centered on corresponding PAS; processing each genomic sub-sequence of the plural genomic sequences, with a corresponding neural network of plural neural networks; supplying plural outputs of the plural neural networks to an interaction layer that includes plural forward Bidirectional Long Short Term Memory Network (Bi-LSTM) cells and plural backward Bi-LSTM cells, wherein each pair of a forward Bi-LSTM cell and a backward Bi-LSTM cell uniquely receives a corresponding output, of the plural outputs, from a corresponding neural network; and generating a scalar value for each PAS, based on an output from a corresponding pair of the forward Bi-LSTM cell and the backward Bi-LSTM cell.

### CROSS-REFERENCE TO RELATED APPLICATIONS

This application claims priority to U.S. Provisional Patent Application No. 62/851,898, filed on May 23, 2019, entitled “DEERECT-APA: ALTERNATIVE POLYADENYLATION SITE USAGE PREDICTION THROUGH DEEP LEARNING,” the disclosure of which is incorporated herein by reference in its entirety.

### BACKGROUND

### Technical Field

Embodiments of the subject matter disclosed herein generally relate to a system, method, and neural network for predicting a usage level of the alternative polyadenylation site, and more particularly, to simultaneously quantitatively predicting the usage of all alternatives polyadenylation sites for a given gene by using deep learning methodologies.

### Discussion of the Background

In eukaryotic cells' genes 100, as illustrated in FIG. 1, the termination of Pol II transcription involves a 3′-end cleavage at 3′-UTR 110 followed by the addition of a poly(A) tail 112, a process termed as “polyadenylation.” The gene 100 also has a cap 102, 5′ UTR 104, and a coding sequence 106. The poly(A) tail consists of multiple adenosine monophosphates, i.e., it is a stretch of RNA that has only adenine bases. In eukaryotes, the polyadenylation is part of the process that produces mature messenger RNA (mRNA) for translation.

The process of polyadenylation begins as the transcription of a gene terminates. The 3′-most segment of the newly made pre-mRNA is first cleaved off by a set of proteins. These proteins then synthesize the poly(A) tail at the RNA's 3′ end. In some genes, these proteins add a poly(A) tail at one of several possible sites. Therefore, polyadenylation can produce more than one transcript from a single gene (alternative polyadenylation).

Often, one gene 100 could have multiple polyadenylation sites (PAS). The so-called alternative polyadenylation (APA) could generate from the same gene locus different transcript isoforms with different 3′-UTRs and sometimes even different protein coding sequences. The diverse 3′-UTRs generated by APA may contain different sets of cis-regulatory elements, thereby modulating the mRNA stability, translation, subcellular localization of mRNAs, or even the subcellular localization and function of the encoded proteins. It has been shown that dysregulation of APA could result in various human diseases. Thus, knowing the PAS and their usage probabilities helps in determining the likelihood of a potential disease.

APA is regulated by the interaction between cis-elements located in the vicinity of PAS and the associated trans-factors. The most well-known cis-element that defines a PAS is the hexamer AAUAAA and its variants are located 15-30nt upstream of the cleavage site, which is directly recognized by the cleavage and polyadenylation specificity factor (CPSF) components: CPSF30 and WDR33. Other auxiliary cis-elements located upstream or downstream of the cleavage site include upstream UGUA motifs bound by the cleavage factor Im (CFIm) and downstream U-rich or GU-rich elements targeted by the cleavage stimulation factor (CstF). The usage of individual PAS for a multi-PAS gene depends on how efficiently each alternative PAS is recognized by these 3′ end processing machineries, which is further regulated by additional RNA binding proteins (RBPs) that could enhance or repress the usage of distinct PAS signals through binding in their proximity.

In addition, the usage of alternative PAS is mutually exclusive. In particular, once an upstream PAS is utilized, all the downstream ones would have no chance to be used no matter how strong their PAS signals are. Therefore, proximal PAS, which are transcribed first, have positional advantage over the distal competing PAS. Indeed, it has been observed that the terminal PAS more often contain the canonical AAUAAA hexamer, which is considered to have a higher affinity than the other variants, which possibly compensates for their positional disadvantage.

There has been a long-standing interest in predicting PAS based on genomic sequences using purely computational approaches. The so-called “PAS recognition problem” aims to discriminate between nucleotide sequences that contain a PAS and those that do not. A variety of hand-crafted features have been proposed and statistical learning algorithms, e.g., random forest (RF), support vector machines (SVM) and hidden Markov models (HMM), are then applied on these features to solve the binary classification problem [1-3]. Very recently, researchers started investigating the “PAS quantification problem”, which aims to predict a score that represents the strength of a PAS [4, 5]. However, the quantification problem is much more difficult than the recognition one.

Recent developments in deep learning have made great improvements on many tasks, for example, to bioinformatics tasks such as protein-DNA binding, RNA splicing pattern prediction, enzyme function prediction, Nanopore sequencing, and promoter prediction. Deep learning is favored due to its automatic feature extraction ability and good scalability with large amount of data. As for the polyadenylation prediction, deep learning models have been applied on the PAS recognition problem and they outperformed existing feature-based methods by a large margin [6].

Recently, deep learning models have also been applied on the PAS quantification problem, where Polyadenylation Code [4] was developed to predict the stronger one from a given pair of two competing PAS. Very recently, another model, DeepPASTA [5] has been proposed. DeepPASTA contains four different modules that deal with both the PAS recognition problem and the PAS quantification problem. Similar to the Polyadenylation Code, the DeepPASTA also casts the PAS quantification problem into a pairwise comparison task.

Thus, there is a need for a new system and method that are capable of quantitatively predicting the usage of all the competing PAS from a same gene simultaneously, regardless of the number of possible PAS.

### BRIEF SUMMARY OF THE INVENTION

According to an embodiment, there is a method for calculating usage of all alternative polyadenylation sites (PAS) in a genomic sequence. The method includes receiving plural genomic sub-sequences centered on corresponding PAS; processing each genomic sub-sequence of the plural genomic sequences, with a corresponding neural network of plural neural networks; supplying plural outputs of the plural neural networks to an interaction layer that includes plural forward Bidirectional Long Short Term Memory Network (Bi-LSTM) cells and plural backward Bi-LSTM cells, wherein each pair of a forward Bi-LSTM cell and a backward Bi-LSTM cell uniquely receives a corresponding output, of the plural outputs, from a corresponding neural network; and generating a scalar value for each PAS, based on an output from a corresponding pair of the forward Bi-LSTM cell and the backward Bi-LSTM cell.

According to another embodiment, there is a computing device for calculating usage of all alternative polyadenylation sites (PAS) in a genomic sequence. The computing device includes an interface configured to receive plural genomic sub-sequences centered on corresponding PAS; and a processor connected to the interface and configured to process each genomic sub-sequence of the plural genomic sequences, with a corresponding neural network of plural neural networks; supply plural outputs of the plural neural networks to an interaction layer that includes plural forward Bidirectional Long Short Term Memory Network (Bi-LSTM) cells and plural backward Bi-LSTM cells, wherein each pair of a forward Bi-LSTM cell and a backward Bi-LSTM cell uniquely receives a corresponding output of the plural outputs; and generate a scalar value for each PAS, based on an output from a corresponding pair of the forward Bi-LSTM cell and the backward Bi-LSTM cell.

According to still another embodiment, there is a neural network system for calculating usage of all alternative polyadenylation sites (PAS) in a genomic sequence. The system includes plural neural networks configured to receive plural genomic sub-sequences centered on corresponding PAS, wherein the plural neural networks are configured to process the genomic sub-sequences such that each neural network processes only a corresponding genomic sub-sequence of the genomic sequence; an interaction layer configured to receive plural outputs of the plural neural networks, wherein the interaction layer includes plural forward Bidirectional Long Short Term Memory Network (Bi-LSTM) cells and plural backward Bi-LSTM cells, and wherein each pair of a forward Bi-LSTM cell and a backward Bi-LSTM cell uniquely receives a corresponding output of the plural outputs; and an output layer configured to generate a scalar value for each PAS, based on an output from a corresponding pair of the forward Bi-LSTM cell and the backward Bi-LSTM cell.

### BRIEF DESCRIPTION OF THE DRAWINGS

For a more complete understanding of the present invention, reference is now made to the following descriptions taken in conjunction with the accompanying drawings, in which:

FIG. 1 illustrates a gene having a PAS;

FIG. 2 illustrates an architecture of a neural network system configured to quantitatively predict PAS usages in a gene;

FIGS. 3A to 3D illustrate various designs of a base network used in the neural network system for handling the PAS;

FIG. 4 illustrates various features used in a handcrafted feature extractor with fully-connected layers;

FIG. 5 shows a list of features used in the handcrafted feature extractor with fully-connected layers and their corresponding dimensions;

FIG. 6 illustrates in more detail the architecture of the neural network system used to quantitatively predict PAS usages in a gene;

FIG. 7 illustrates averaged comparison accuracy across 5-fold cross validation of two ablated models and one full model;

FIG. 8 illustrates the hyperparameters for three novel models having different base networks;

FIG. 9 illustrates the performance of the novel neural network system on parental and F1 mouse fibroblast datasets;

FIG. 10 illustrates the performance of the neural network system when compared with traditional models for the previous two different datasets;

FIG. 11 illustrates an improvement of the neural network system over other traditional methods and the improvement is tested to be statistically significant;

FIG. 12 illustrates the improvement of the neural network system over the other two methods for various tissues;

FIGS. 13A to 13E illustrate the prediction of the neural network system for a given gene;

FIGS. 14A and 14B illustrate the effect of genetic mutations, more specifically, substitution mutations, on the usage of PAS as identified by transcriptomic sequencing;

FIGS. 15A to 15D illustrate predictions of the neural network system for another gene which shows that the DeeReCT-APA model can predict the effect of insertion mutations correctly;

FIG. 16 illustrates Pearson correlations (and their p-values) between two quantities at different minimum allelic usage difference for the neural network system and a traditional method;

FIGS. 17A to 17D illustrate visualization examples of the learned convolutional filters of the neural network system;

FIG. 18 is a flowchart of a method for calculating usage of all alternative PAS in a genomic sequence; and

FIG. 19 illustrates a computing system in which the method or the neural network systems discussed herein can be implemented.

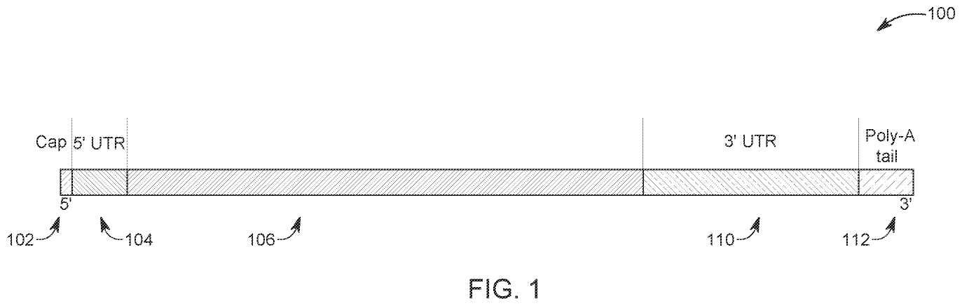

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel deep learning model, called herein DeeReCT-APA (Deep Regulatory Code and Tools for Alternative Polyadenylation), for the PAS quantification problem is introduced. The DeeReCT-APA model can simultaneously, quantitatively predict the usage of all the competing PAS from a same gene, regardless of the number of PAS. The model is trained and evaluated based on a dataset from a previous study, which consists of a genome-wide PAS measurement of two different mouse strains (C57BL/6J (BL) and SPRET/EiJ (SP)), and their F1 hybrid. After training the novel model on the dataset, the novel model is evaluated based on a number of criteria. The novel model demonstrates the necessity of simultaneously modeling the competition among multiple PAS. The novel model is found to predict the effect of genetic variations on APA patterns, visualize APA regulatory motifs and potentially facilitate the mechanistic understanding of APA regulation.

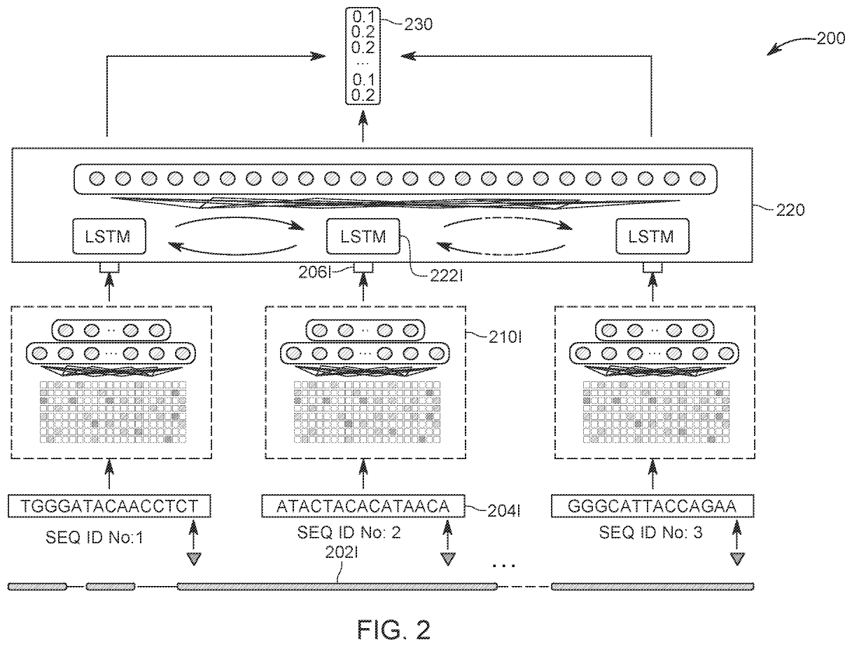

The novel DeeReCT-APA model (also called method herein) is based on a deep learning architecture 200 (or neural network system), as shown in FIG. 2. The neural network system 200 includes plural base neural networks 210I or base networks, Base-Net, one for each competing PAS 202I, and upper-level interaction layers 220, where 1 is an integer that corresponds to the total number of the PAS. In one application, each base network 210I takes a 455nt long genomic DNA sequence 204I, (called herein sub-sequence) centered around one competing PAS cleavage site 202I, as input, and produces as output, a vector 206I, which can be interpreted as the distilled features of that sub-sequence 204I. For example, the distilled feature may be a sequence motif, which is a nucleotide or amino-acid sequence pattern that is widespread and has, or is conjectured to have, a biological significance. In another application, the same base network 210I is used throughout the neural network system 200. However, in another application, it is possible to use different base networks for different PAS. The results of the upper-level interaction layers 220 is used to predict, in the output layer 230, the usage level of each PAS, as schematically illustrated in FIG. 2. The components of the neural network system 200 are now discussed in more detail.

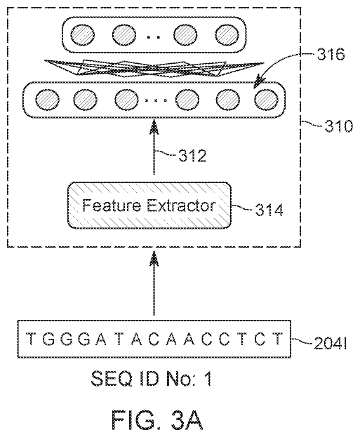

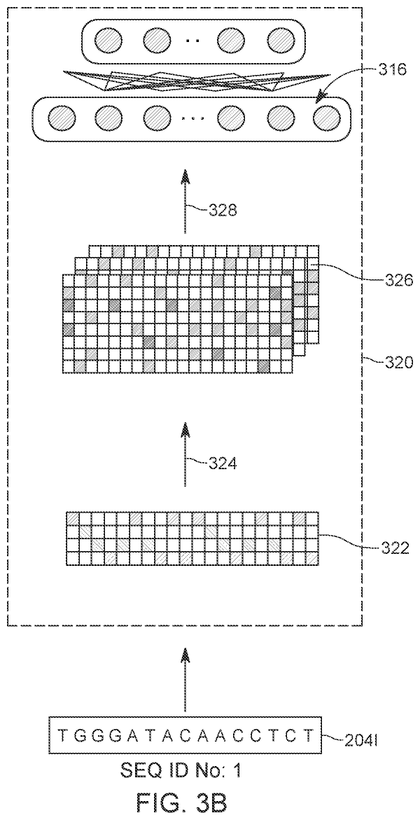

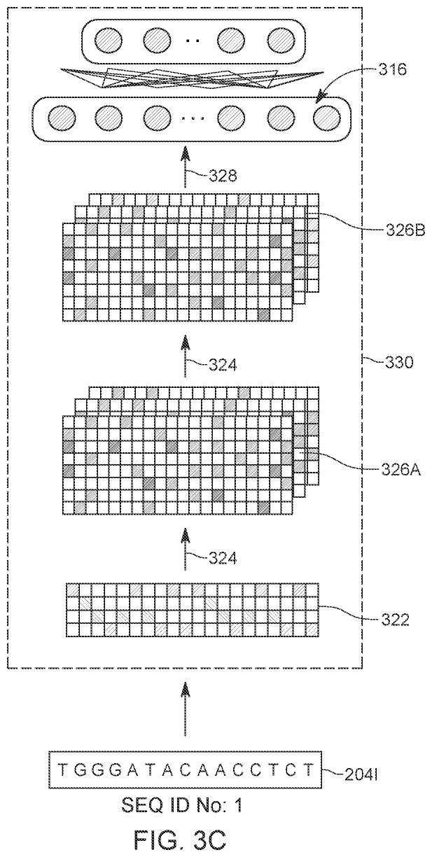

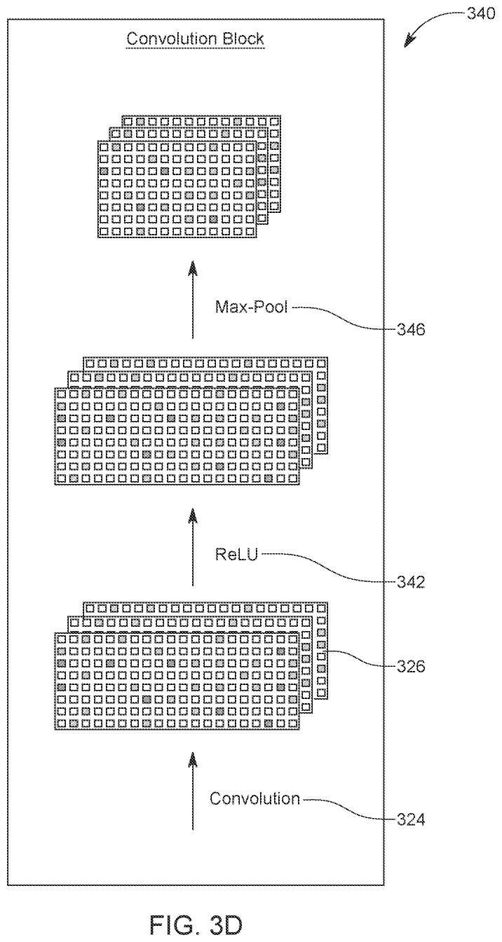

There are two types of base networks that can be used in the architecture 200, and these base networks include: (1) a hand-engineered feature extractor 310, as schematically illustrated in FIG. 3A, or (2) a convolutional neural network (CNN) 320 or 330, as schematically illustrated in FIGS. 3B and 3C, respectively.

The hand-engineered feature extractor 310 shown in FIG. 3A receives as input the sub-sequence 204I, extracts one or more features 312 from the sub-sequence 204I using the feature extraction layer 314, and provides the extracted feature 312 to a fully connected layer 316.

The CNN 320 in FIG. 3B receives the sub-sequence 204I and applies a one-hot encoding 322 to it. The encoded data is then convolved in a convolution step 324, which results in convolved data 326. The convolved data is flattened in step 328 and then applied to the fully connected layer 316. The CNN 330 shown in FIG. 3C is different from the CNN 320 in the sense that two or more convolution steps 324, with different convolution blocks 326A and 326B, are applied to the input data instead of a single convolution step as in the CNN 320.

The three different base network configurations are deep neural network architectures. In one embodiment, the base network in FIG. 3A is based on a handcrafted feature extractor with fully-connected layers (Feature-Net 310), the base network in FIG. 3B is based on a single 1D convolution layer (Single-Cony-Net 320), and the base network in FIG. 3C is based on multiple 1D convolution layers (Multi-Cony-Net). Single-Cony-Net 320 and Multi-Cony-Net 330 are two convolutional neural network (CNN) structures for the Base-Net. The Single-Cony-Net consists of only one layer of the 1D convolutional block (324 and 326) and takes directly the one-hot encoded sequences as input. The convolutional block 324/326, called herein block 340, which is shown in more detail in FIG. 3D, has a number of convolution filters which become automatically-learned feature extractors after training. A rectified linear unit (ReLU) 342 is used as the activation function. The max-pooling operation 346 after that allows only values from highly-activated neurons to pass to the upper fully-connected layers. The three operations of the convolution block 340 include the convolution operation 324, the ReLU 342, and the max-pooling 346. While the Single-Cony-Net 320 uses one convolution block 340, the Multi-Cony-Net 330 uses two convolution blocks 340 before the fully-connected layers 316. The increased depth of the network makes it possible for the network to learn more complex representations.

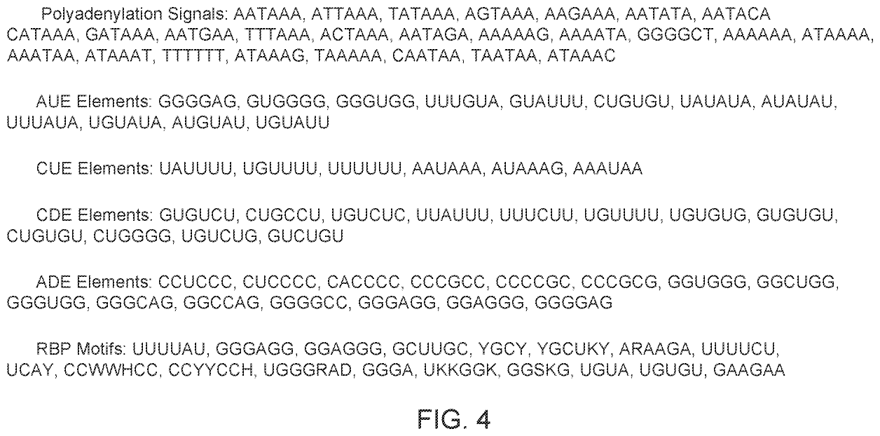

The Feature-Net 310 only consists of multiple fully-connected layers 316 and takes as input multiple types of features extracted from the sub-sequences 204I of interest. The features, described in more detail in [4], may include polyadenylation signals, auxiliary upstream elements, core upstream elements, core downstream elements, auxiliary downstream elements, RNA-binding protein motifs, as well as 1-mer, 2-mer, 3-mer and 4-mer features, which are illustrated in FIGS. 4 and 5. Each feature value corresponds to the occurrence of each motif. The extracted features are then z-score normalized.

The output of the lower-level base network 210I is then passed to the upper-level interaction layers 220, which computationally model the process of choosing the competing PAS. The interaction layers 220 of the DeeReCT-APA neural network system 200 are based in this embodiment on the Long Short Term Memory Networks (LSTM) [7], which are designed to handle natural language processing and can naturally handle sentences with an arbitrary length, which makes them suitable for handling any number of alternative PAS from a same gene locus. The interaction layers then output the percentage values of all the competing PAS of the gene, as illustrated in FIG. 2.

The utilization of alternative PAS is intrinsically competitive. On one hand, as a multi-PAS gene is transcribed, any one of its PAS along the already transcribed region is possible to be used. However, if one of the PAS has already been used, it will make other PAS impossible to be chosen. On the other hand, given that the same polyadenylation machinery is used by all the alternative PAS, such competition of resources also contributes to the competitiveness of this process.

Previous work [4, 5] in polyadenylation usage prediction did not take this important point into account. Both models introduced in [4, 5], Polyadenylation Code and DeepPASTA (tissue-specific relatively dominant poly(A) sites prediction model), can only handle two PAS at a time, ignoring the competition with the other PAS. To overcome this limitation of the traditional methods, the DeeReCT-APA neural network system considers all the competing PAS at the same time and simultaneously take as input all the PAS in a gene, thus jointly predicting the usage levels of all of PAS. This fundamental difference between the existing models and the DeeReCT-APA model makes this model advantageous.

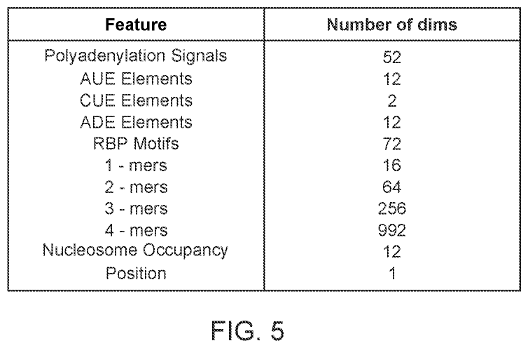

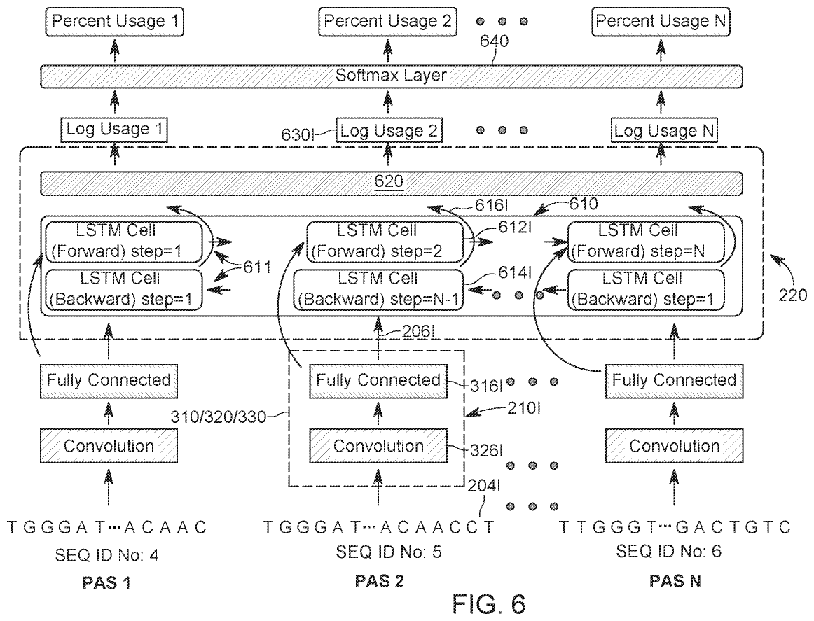

To fulfil this simultaneous condition, the interaction layers 220 above the base networks 210I are configured to model the interaction between different PAS. In neural networks, a way to model interactions among inputs is to introduce a recurrent neural network (RNN) layer, which can capture the interdependencies among inputs corresponding to each time step. In this embodiment, the LSTM layer [7] was selected to be the foundation of the interaction layers 220. The LSTM layer is a type of RNN that has hidden memory cells which are able to remember a state for an arbitrary length of time steps. To fit into the PAS usage level prediction task, each time step of the LSTM layer corresponds to one PAS, at which the LSTM takes the extracted features of that PAS from the lower-level base network 210I. As there is both an influence from upstream PAS to downstream PAS and vice versa, in this embodiment, the DeeReCT-APA model uses a bidirectional LSTM (Bi-LSTM), in which one LSTM's time step goes from the upstream PAS to the downstream one and the other from the downstream to the upstream, as shown by the arrows 611 in FIG. 6.

More specifically, FIG. 6 shows that for each PAS (PAS 1 to PAS N), a corresponding sub-sequence 204I is received as input by the base network 210I, which may be implemented as one of the Single-Cony-Net 320, or the Multi-Cony-Net 330, or the Feature-Net 310. The base network 210I is shown in the figure as being the Single-Cony-Net 320, which includes the convolution block 326I and the fully connected layers 316I. The output from each base network 210I is then supplied to the interconnection layers 220. The interconnection layers 220 are configured to include the Bi-LSTM layer 610 and a linear transformation layer 620. The Bi-LSTM layer 610 includes a plurality of forward LSTM cells 612I and a plurality of backward LSTM cells 614I, where I indicates the step and varies between 1 and N, with N being an integer. For each base network 210I, the interconnection layers 220 includes a corresponding forward LSTM cell 612I and a backward LSTM cell 614I. The forward LSTM cells 612I are linked to each other in a sequence, from 1=1 to 1=N and each base network 210I is uniquely connected to a corresponding forward LSTM cell 612I so that an output from the base network 210I is feed directly to the corresponding forward LSTM cell 612I. The backward LSTM cells 614I are linked to each other in another sequence, from 1=N to 1=1 and each of the backward LSTM cells 614I is also uniquely connected to the output of the base network 210I. Thus, each pair of a forward LSTM cell 612 and a backward LSTM cell 614 is uniquely connected to a corresponding base network 210I, and receives only the output 206I from that base network.

The outputs of the forward LSTM cell 612I and the backward LSTM cell 614I at the same PAS are then concatenated and sent as output 616I to the upper fully-connected layer 620. The fully-connected layer 620 transforms the LSTM output to a scalar value representing the log-probability 630I of that PAS to be used. After the log-probabilities of all competing PAS pass through a final SoftMax layer 640, they are transformed to properly normalized percentage scores, which sum up to one, representing the probability of each PAS of being chosen. Although the DeepPASTA method also contains a Bi-LSTM component, their Bi-LSTM layer is configured to process the sequence of one of the two competing PAS that are given as input. The time steps of the Bi-LSTM in the DeepPASTA method correspond to different positions in one particular sequence rather than to different PAS as in the case for the configuration of FIG. 6, and therefore, the Bi-LSTM layer in the DeepPASTA model is not used to model the interactions between different PAS, which is the case in the DeeReCT-APA model.

In other words, the DeeReCT-APA model shown in FIGS. 2 and 6 takes all PAS of a gene, as a whole, and predicts the usage level of each PAS as accurately as possible while no other previous model considers more than two PAS at a same time. Therefore, at one time, the DeeReCT-APA model takes all PAS in a gene as input. Considering that the number of PAS within a gene is not a constant, the DeeReCT-APA model is configured to take inputs of a variable length. Since most genes have a small number of PAS, the DeeReCT-APA model does not pad all the genes with dummy PAS to make them of the same length, as this will make the model highly inefficient. Instead, the interaction layers are designed in a way that it can take an arbitrary number of base networks 210I. For example, it is possible to implement customized layers in the deep learning library to scan from the first PAS to the last one in order to deal with the variable-length input.

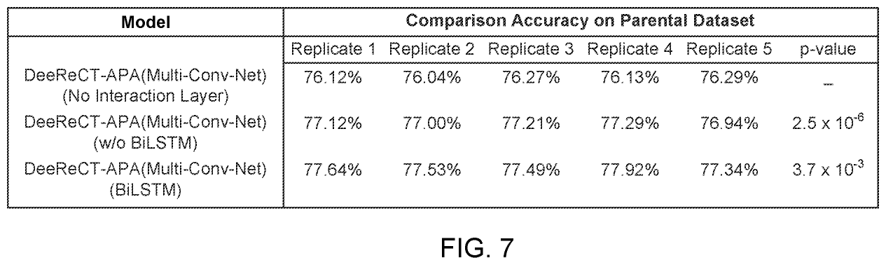

Two experiments were performed with the DeeReCT-APA model to evaluate the effect of the Bi-LSTM interaction layer 610. The first experiment removes the Bi-LSTM layer from the interconnected layers 220 and only keeps the fully-connected layer 620 and the SoftMax layer 640. In this scenario, the modified network still simultaneously considers all PAS of a gene, but with a non-RNN interaction layer 220. The second experiment removes the interaction layer 220 altogether and uses a comparison-based training (like in Polyadenylation Code) to train the base networks 210I. As shown in the table of FIG. 7, the usage of the Bi-LSTM interaction layer positively contributes to the performance of the DeeReCT-APA model. FIG. 7 compares the performance of (1) the DeeReCT-APA model with the Multi-Cony-Net and without the interaction layer, to (2) the DeeReCT-APA model with the interaction layer but without the Bi-LSTM layer, and (3) the DeeReCT-APA model with the interaction layer and with the Bi-LSTM layer. In terms of all metrics, both the usage of the interaction layer and the Bi-LSTM layer improve the performance of the model.

A genome-wide PAS quantification dataset derived from fibroblast cells of C57BL/6J (BL) and SPRET/EiJ (SP) mouse, as well as their F1 hybrid is obtained from a previous study [8]. In the F1 cells, the two alleles have the same trans environment and the PAS usage difference between two alleles can only be due to the sequence variants between their genome sequences, making it a valuable system for APA cis-regulation study. Apart from APA, this kind of systems have also been used in the study of alternative splicing and translational regulation.

The detailed description of the sequencing protocol and data analysis procedure can be found in [8]. As a brief summary, the study uses fibroblast cell lines from BL, SP and their F1 hybrids. The total RNA is extracted from fibroblast cells of BL and SP undergoes 3′-Region Extraction and Deep Sequencing (3′READS) to build a good PAS reference of the two strains. The 3′-mRNA sequencing is then performed in all three cell lines to quantify those PAS in the reference. In the F1 hybrid cell, reads are assigned to BL and SP alleles according to their strain specific SNPs. The PAS usage values are then computed by counting the sequencing reads assigned to each PAS. The sequence centering around each PAS cleavage site (448nt in total) is extracted and undergoes feature extraction or one-hot encoding before training the model. The extracted features are then inputted to the Feature-Net 310, while the one-hot encoded sequences are inputted to the Single-Cony-Net 320 and the Multi-Cony-Net 320.

The DeeReCT-APA model is trained based on the parental BL/SP PAS usage level dataset. For the F1 hybrid data, however, it was chosen to start from the pre-trained parental model (for which either the BL parental model or the SP parental model were used and the results are shown separately) and fine-tune the model on the F1 dataset. This is because, due to the read assignment problem, the usage of many PAS in F1 cannot be unambiguously characterized by 3′-mRNA sequencing [8]. As a result of this process, the F1 dataset does not contain an enough number of PAS to train the DeeReCT-APA model from scratch. At the training stage, genes are randomly selected from the training set and the sequences of their PAS flanking regions are fed into the network. Each sequence of PAS in a gene passes through one Base-Net 210I. The parameters of the Base-Net 210I that are responsible for each PAS are all shared. Each Base-Net 210I then outputs a vector representing the distilled features for each PAS, which is then sent to the interaction layers 220. The interaction layers 220 generate a percentage score of each PAS of this gene. Cross-entropy loss between the predicted usage and the actual usage is used as the training target. During back-propagation, the gradients are back-propagated through the passage originated from each PAS. As the model parameters are shared between the base networks 210I, the gradients are then summed up to update the model parameters.

Several techniques are used to reduce the overfitting: (1) Weight decay is applied on weight parameters of CNN and all fully-connected layers; (2) Dropout is applied on the Bi-LSTM layer; (3) The training is stopped as soon as the mean absolute error of the predicted usage value does not improve on the validation set; (4) While fine-tuning the model on the F1 dataset, a learning rate that is about 100 times smaller than the one used when training from scratch is used.

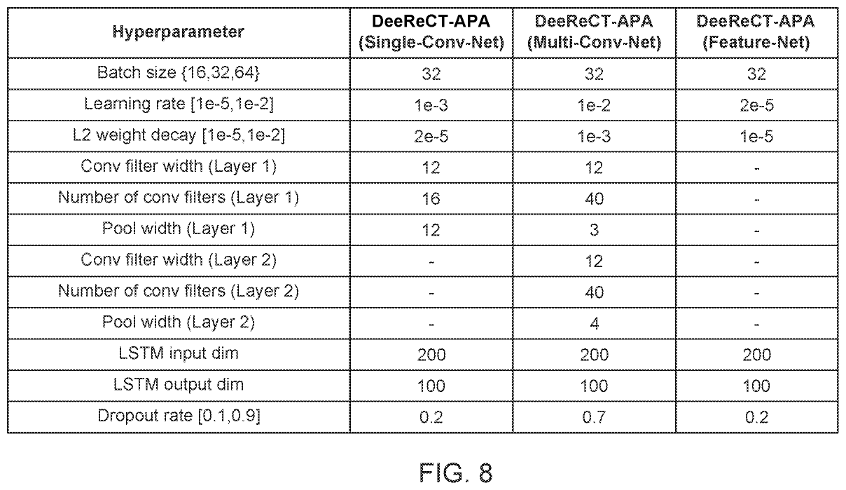

The neural network system 200 is trained with the adaptive moment estimation (Adam) optimizer. A detailed list of hyperparameters used for the training is shown in FIG. 8. For the hyperparameters that are sampled randomly, the ranges (in brackets) or sets (in braces) that they are sampled from are shown. The hyperparameters that are not applicable for a specific model are denoted by “-”. The neural network system 200 is constructed in one application using the PyTorch deep learning framework and utilizes one NVIDIA GeForce GTX 980 Ti as the GPU hardware platform.

To evaluate the performance of the DeeReCT-APA model, a 5-fold cross-validation is performed at the gene level using all the genes in the dataset for each strain. That is, if a gene is selected as a training (testing) sample, all of its PAS are in the train (test) set. At each time, four folds are used for training and the remaining one is used for testing. To make a fair comparison with the existing methods Polyadenylation Code and DeepPASTA previously discussed, the two models are also trained (fine-tuned) and their model parameters are optimized on the parental and F1 datasets introduced above.

The following measures are used for evaluating the DeeReCT-APA model against its baseline and also against state-of-the-art models: mean absolute error (MAE), comparison accuracy, highest usage prediction accuracy, and average Spearman's correlation. The MAE metric is defined as the mean absolute error of the usage prediction of each PAS, which is given by:

$$MAE = \frac{1}{M} {\sum }_{i = 1}^{M} \vert {p}_{i} - {t}_{i} \vert , \qquad ( 1 )$$

where pᵢ stands for the predicted usage, tᵢ stands for the experimentally determined ground truth usage for PAS i and M is the total number of PAS across all genes in the test set. This is the most intuitive way of measuring the performance of the DeeReCT-APA model. However, this measure is not applicable to the Polyadenylation Code or DeepPASTA methods as they do not have quantitative outputs that can be interpreted as the PAS usage values. For the same reason, this measure is not applicable to the DeeReCT-APA model either, when its interaction layers are removed and the comparison-based training is used.

The Comparison Accuracy is based on the Pairwise Comparison Task, which is defined as listing all the pairs of PAS in a given gene and keeping only those pairs with PAS usage level difference greater than 5%. The model is asked to predict which PAS in the pair is of the higher usage level. The accuracy is defined as

$$Comparison Accuracy = \frac{\# Pairs Correctly Predicted}{\# All Pairs} . \qquad ( 2 )$$

The primary reason that this metric is used to compare the DeeReCT-APA model with the Polyadenylation Code and DeepPASTA models, is that these traditional models were designed for predicting which one is stronger between the two competing PAS.

The Highest Usage Prediction Accuracy measure aims to test the model's ability of predicting which PAS is of the highest usage level in a single gene. For this measure, all the genes are selected that have their highest PAS usage level greater than their second highest one by at least 15% in the test set for evaluation. For the DeeReCT-APA model, the predicted usage in percentage is used for ranking the PAS. For the Polyadenylation Code and DeepPASTA models, as they do not provide a predicted value in percentage, the logit value before the SoftMax layer is used. The logit values, though not in the scale of real usage percentage values, can at least give a ranking of different PAS sites. The highest usage prediction accuracy is the percentage of genes whose highest-usage PAS are correctly predicted.

The Averaged Spearman's Correlation is defined as follows. The predicted usage levels by each model is converted into a ranking of PAS sites in that gene. The Spearman's correlation is computed between the predicted ranking and ground truth ranking. The correlation values for all genes are then averaged together to give an aggregated score. In other words,

$$Averaged Spearman ’ s Correlation = \frac{1}{N} {\sum }_{i = 1}^{N} \frac{{\sum }_{p = 1}^{{P}_{i}} ( p {r}_{ip} - \overline{{pr}_{l}} ) ( g {r}_{ip} - \overline{{gr}_{l}} )}{\sqrt{{\sum }_{p = 1}^{{P}_{i}} {( p {r}_{ip} - \overline{{pr}_{l}} )}^{2}} \sqrt{{\sum }_{p = 1}^{{P}_{i}} {( g {r}_{ip} - \overline{{gr}_{l}} )}^{2}}} \qquad ( 3 )$$

where N is the total number of genes, Pᵢ is the number of PAS in gene i, prᵢₚ is the predicted rank of PAS p in gene i, grᵢₚ is the ground truth rank of PAS p in gene i, and prᵢ and grᵢ are the averaged predicted and ground truth ranks in gene i, respectively.

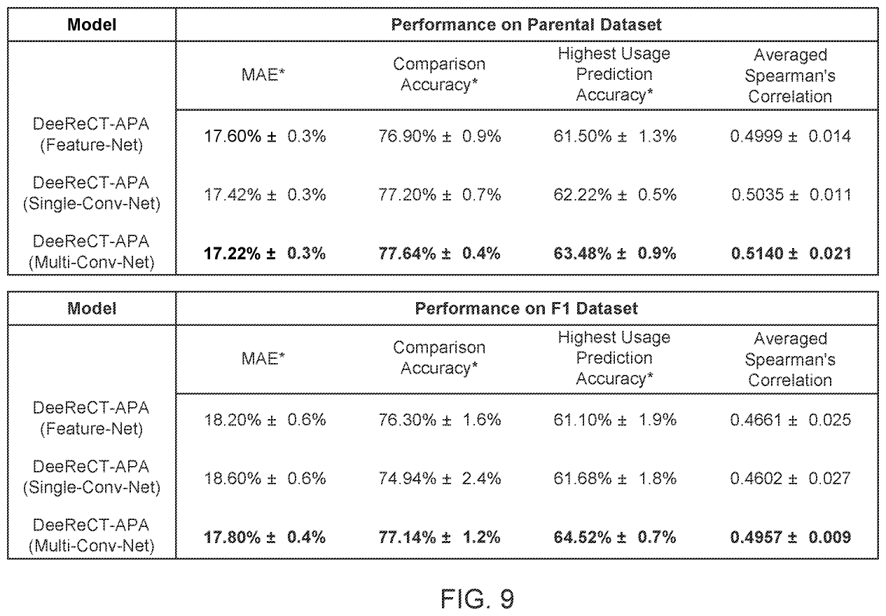

Based on these measures, the performance of various Base-Net designs are first evaluated. The DeeReCT-APA model is evaluated first with the Feature-Net module 310, then with the Single-Cony-Net module 320, and finally with the Multi-Cony-Net 330. As shown in the table of FIG. 9, both on the parental BL dataset and on the F1 dataset, the DeeReCT-APA model with the Multi-Cony-Net performs the best, followed by that with the Single-Cony-Net. This is expected as the deeper neural networks have higher representation learning capacity.

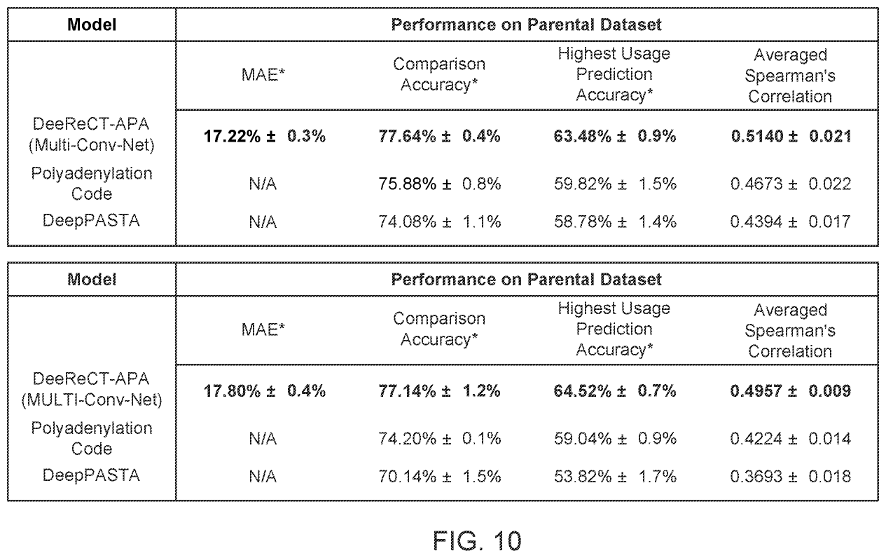

Then, the performance of the DeeReCT-APA model with the Multi-Cony-Net module was compared to the Polyadenylation Code and DeepPASTA. As shown in FIG. 10, both on the parental BL dataset and on the F1 dataset, the DeeReCT-APA model with the Multi-Cony-Net consistently performs best across all four metrics. The standard deviation across the 5-fold cross validation is higher in the F1 dataset than in the parental dataset, indicating the increased instability in F1 prediction, which is probably due to the limited amount of F1 data. As a rather small dataset was used, a very complex model like the DeepPASTA model is prone to overfitting, which is probably the reason why it performs the worst here. Indeed, for the smaller F1 dataset, the DeepPASTA model lags even more behind other methods. Unless otherwise stated, the F1 dataset that is used in the remaining part of this disclosure is the one fine-tuned from the parental BL model, modified to include the training set folds that do not include the gene or PAS to be tested.

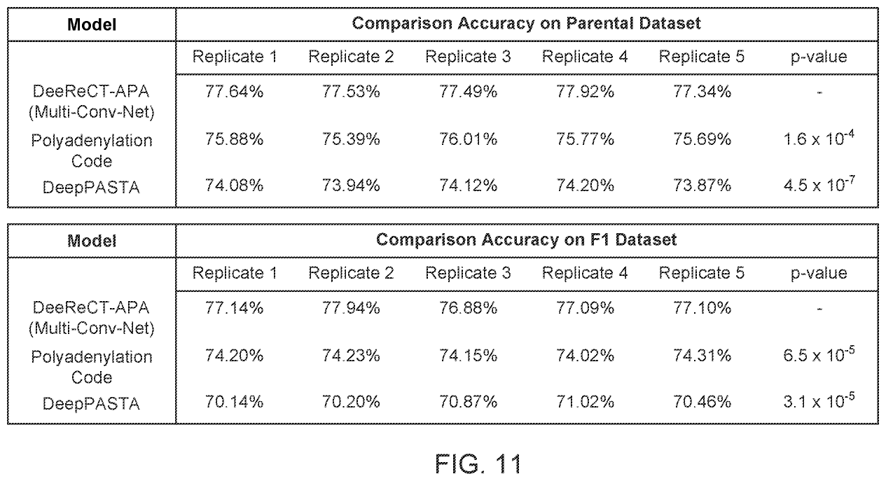

The improvement made by the DeeReCT-APA model is statistically significant in terms of comparison accuracy, even though the performance improvement is not numerically substantial. For this purpose, the experiment is repeated five times, with each repeat having the dataset randomly split in a different way, and the accuracy of the DeeReCT-APA model (using the Multi-Cony-Net), Polyadenylation Code, and DeepPASTA is reported after 5-fold cross validation. The performance of the three tools is then compared with the p-value computed by t-test. As shown in FIG. 11, the improvement of the DeeReCT-APA model over the other two methods is statistically significant.

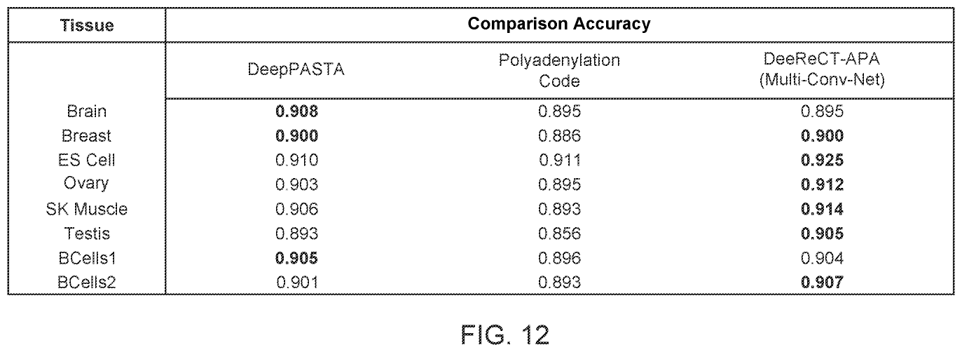

To demonstrate that the results of the above comparison are independent of the datasets, the DeeReCT-APA model was trained and tested on another dataset used in [4]. Because this dataset consists of polyadenylation quantification data from multiple human tissues, the performance (comparison accuracy) of the DeeReCT-APA model is reported for each tissue separately, as illustrated in FIG. 12. The performance metrics of the Polyadenylation Code and DeepPASTA models is adapted from [4] and [5] accordingly. For 6 out of the 8 tissues considered, the DeeReCT-APA model achieves a higher accuracy than the other two methods.

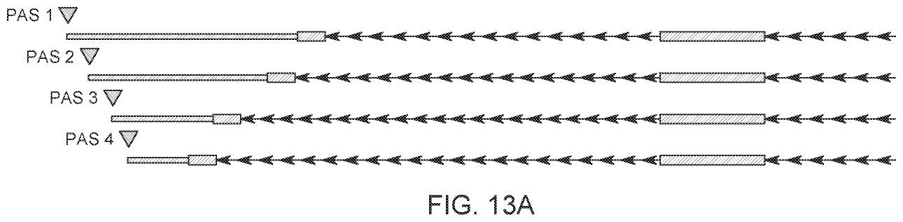

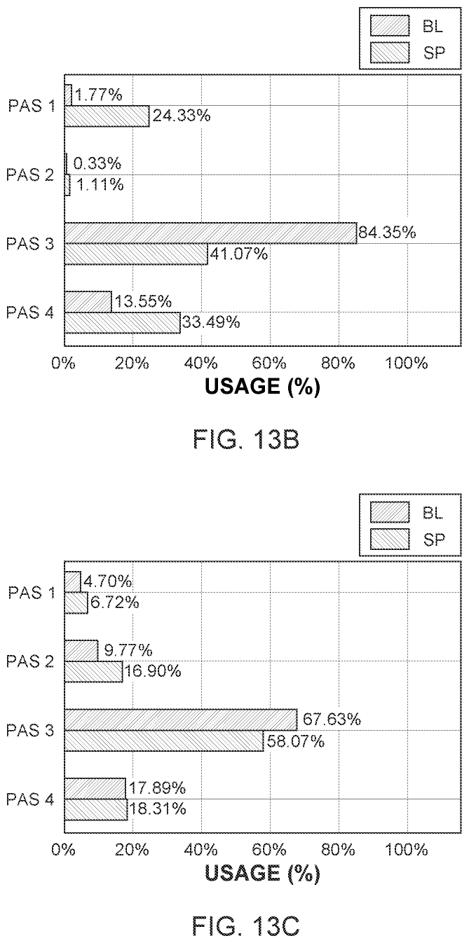

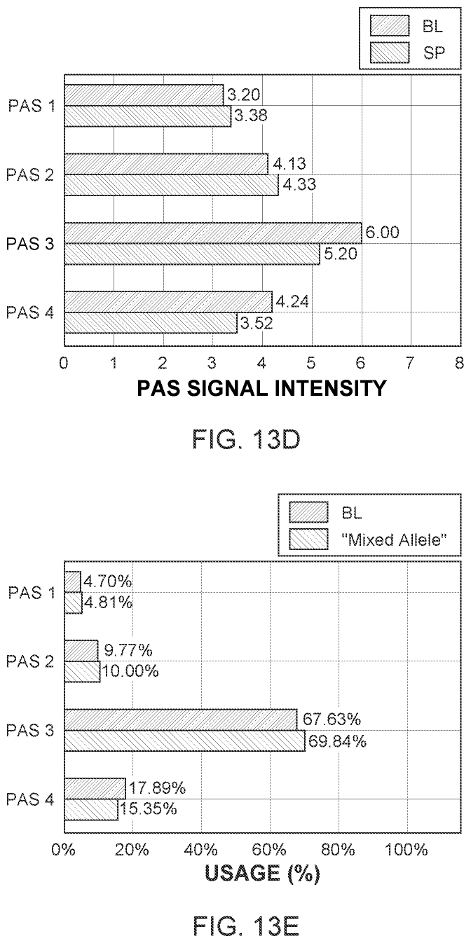

The benefits of jointly modelling all the PAS, as implemented in the DeeReCT-APA model of FIG. 2 is now discussed. To illustrate the DeeReCT-APA model's ability of modeling all PAS of a gene jointly, the gene Srr (Ensembl Gene ID: ENSMUSG00000001323) is used as an example. As shown in FIG. 13A, the gene Srr use four different PAS. FIG. 13B shows the ground truth usage level for the two datasets BL and SP, for the four PAS, FIG. 13C shows the prediction of the DeeReCT-APA model with the Multi-Cony-Net for the F1 hybrid cell for those four PAS, for both its BL allele (BL bars) and SP allele (SP bars), respectively, and FIG. 13D shows the prediction of the Polyadenylation Code for the F1 hybrid cell for those four PAS, for both its BL allele (BL bars) and SP allele (SP bars), respectively. As before, the logits values before the SoftMax layer of the Polyadenylation Code are used as surrogates for the predicted usage values (and therefore not in the range from 0 to 1). As shown in FIGS. 13B to 13D, the prediction of the DeeReCT-APA model (see FIG. 13C) is more consistent with the ground truth (see FIG. 13B) than that of the Polyadenylation Code (see FIG. 13D) and the relative magnitude between the BL allele and SP allele for the prediction of the DeeReCT-APA model in FIG. 13C is correct for all four PAS. In comparison, the Polyadenylation Code model predicted PAS 4 in the BL allele in FIG. 13D shows slightly higher usage than the one in the SP allele whereas both in ground truth and the prediction made by the DeeReCT-APA model, the reverse is true. It is believed in this case that the genetic variants between the BL allele and SP allele in the sequences flanking PAS 4 alone might make the BL allele a stronger PAS than the SP allele because the Polyadenylation Code only considers which one between the two is stronger and predicts the strength of a PAS solely by its own sequence, without considering those of the others. However, when simultaneously considering genetic variations in PAS 1, PAS 2 and PAS 3, which probably have stronger effects, the usage of PAS 4 becomes lower in BL than in SP.

To test this explanation, an in-silico experiment was designed by constructing a hypothetical allele of gene Srr (hereafter referred to as “mixed allele”) that has the BL sequence of PAS 1, PAS 2 and PAS 3, and SP sequence of PAS 4. Then, the Dee ReCT-APA model was asked to predict the usage level of each PAS in the “mixed allele,” where the usage differences between the BL allele and the “mixed allele” should then be purely due to the sequence variants in PAS 4 because the two alleles are exactly the same on the other PAS. As shown in FIG. 13E, consistent with this explanation, the usage level of PAS 4 in the BL allele is indeed higher than that in the “mixed allele.” This example demonstrates the benefit of jointly and simultaneously modeling all the PAS in a gene as in the DeeReCT-APA model.

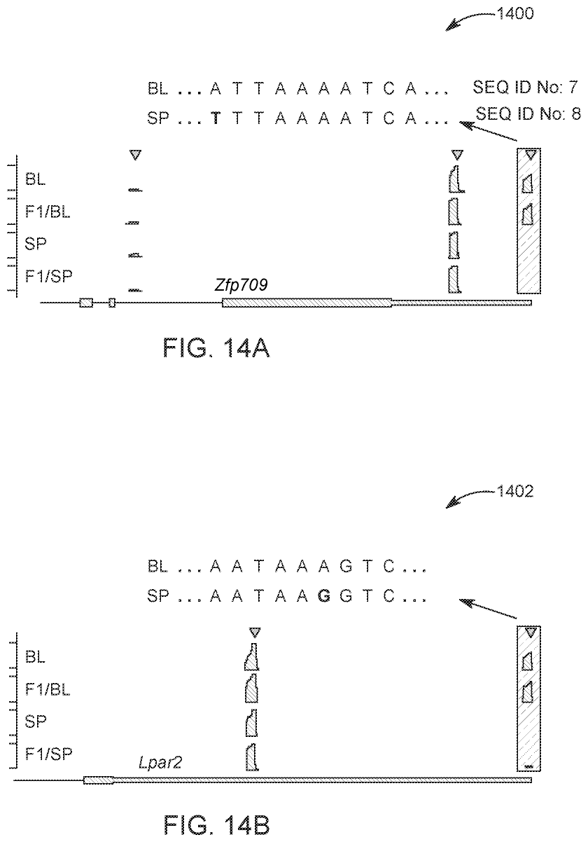

A goal of the DeeReCT-APA model is to determine the effect of sequence variants on the APA patterns. The F1 hybrid dataset chosen here is ideal to test how well such a goal is achieved, since in the F1 cells, the allelic difference in PAS usage can only be due to the sequence variants between their genome sequences. In this regard, FIGS. 14A and 14B show two genes, gene Zfp709 and Lpar2, for which a previous analysis demonstrated that in the distal PAS of gene Zfp709, a substitution (from A to T) in the SP allele relative to the BL allele (see location 1400 in FIG. 14A) disrupted the PAS signal (ATTAAA to TTTAAA). In the distal PAS of gene Lpar2, a substitution (from A to G) in the SP allele relative to the BL allele (see location 1402 in FIG. 14B) disrupted another PAS signal (AATAAA to AATAAG), causing both of them to be of lower usage in the SP allele than in the BL allele.

To check whether the novel DeeReCT-APA model could be used to identify the effects of these variants, a “mutation map” was plotted for the two genes. In brief, for each gene, given the sequence around the most distal PAS (suppose it is of length L), 3L “mutated sequences” were generated. Each one of the 3L sequences has exactly one nucleotide mutated from the original sequence. These 3L sequences are then fed into the model along with other PAS sequences from that gene and the model then predicts usage for all sites and for each of the 3L sequences, separately. The predicted usage values of the original sequence are then subtracted from each of the 3L predictions and plotted in a heatmap, the “mutation map.” The obtained heatmap entries that correspond to the sequence variants between BL and SP are consistent with experimental findings from [8]. In addition, the mutation maps can also show the predicted effect of sequence variants other than those between BL and SP, giving an overview of the effects from all potential mutations.

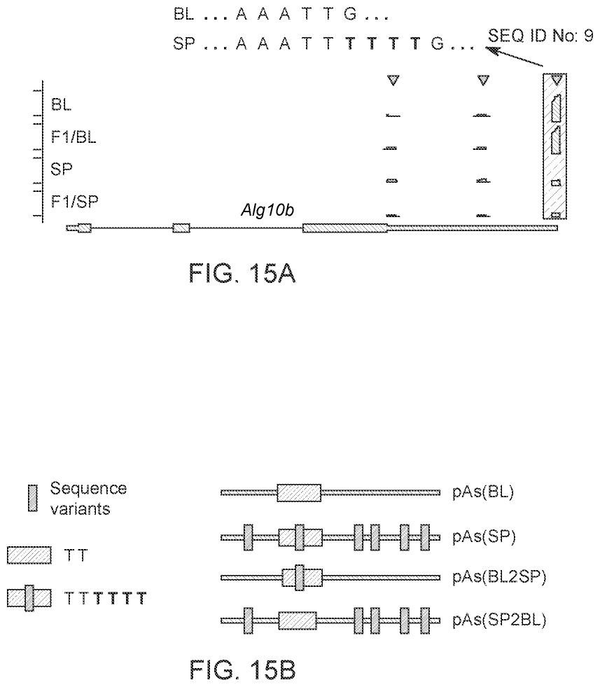

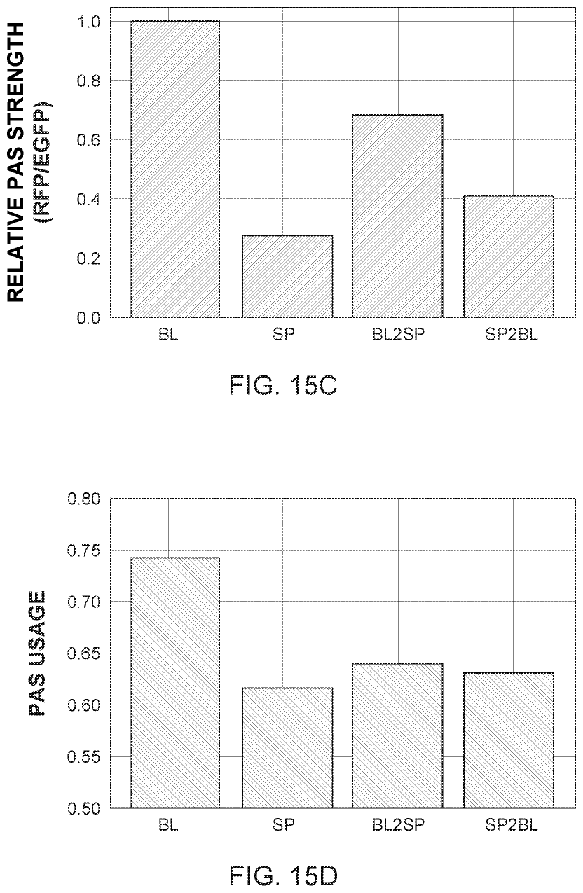

The two examples described above involve sequence variants disrupting PAS signals, which makes the prediction relatively simple. To check whether the Dee ReCT-APA model could be used for the variants with more subtle effects, a third example, gene Alg10b, was selected for some tests. Previous experiments showed that the usage of the most distal PAS of its BL allele is higher than its SP allele, as illustrated in FIG. 15A. Using reporter assays as illustrated in FIG. 15B, it has been demonstrated (see [8]) that an insertion of UUUU in the SP allele relative to the BL allele contributes to this reduction in usage (see FIG. 15C). To check whether the DeeReCT-APA model could reveal such effects, the same four sequence variants shown in FIG. 15B were constructed in silico sequences as in [8]: BL, SP, BL2SP and SP2BL. Together with other PAS of the gene Alg10b, the four sequences are feed to the DeeReCT-APA model, separately. As shown in FIG. 15D, when comparing BL with BL2SP and SP with SP2BL, the DeeReCT-APA model is able to reveal the negative effect of the poly(U) tract.

To globally evaluate the performance of the DeeReCT-APA model on predicting the allelic difference in PAS usage, the predicted allelic difference were compared to the experimentally measured allelic difference in a genome-wide manner. As a baseline control, the same was performed for the prediction made by the Polyadenylation Code where logit values before the SoftMax layer were used as surrogates for the predicted allelic difference in PAS usage. Here, the F1 dataset fine-tuned from the BL parental model is used. It is worth noting that this is a very challenging task because the training data do not well represent the complete landscape of genetic mutations. That is, the BL dataset only contains invariant sequences from different PAS, and the F1 dataset contains a limited number of genetic variants.

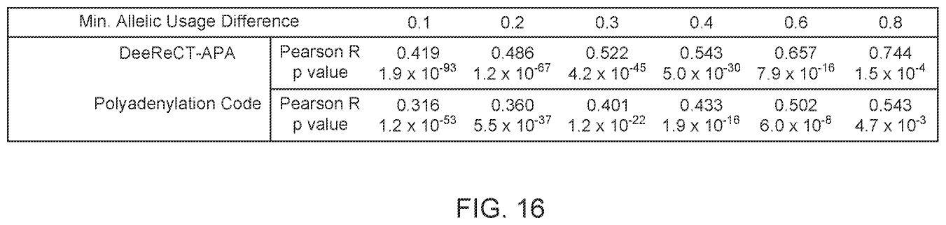

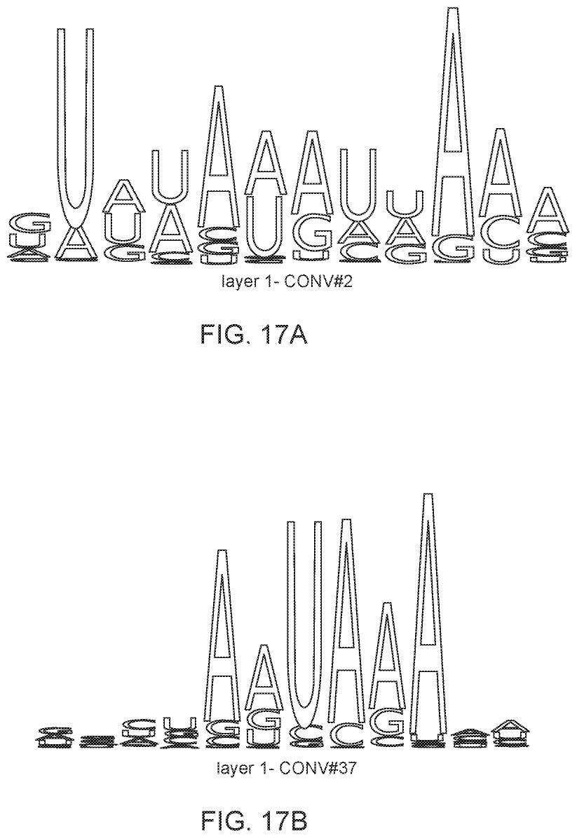

The Pearson correlation between the experimentally measured allelic usage difference and the ones predicted by the two models was computed as illustrated in the table in FIG. 16. The obtained data shows that the DeeReCT-APA model outperforms the Polyadenylation Code. The Pearson correlation values were evaluated using six subsets of the test set, each filtering out PAS with allelic usage difference less than 0.1, 0.2, 0.3, 0.4, 0.6, 0.8, respectively, as shown in FIG. 16. When the allelic usage difference is small, their relative magnitudes are more ambiguous and the experimental measurement of their allelic usage difference (used here as ground truth) are less confident. Indeed, with the increasing allelic difference, the prediction accuracy increased for both the DeeReCT-APA model and the Polyadenylation Code. Further, it is observed that in all these groups, the DeeReCT-APA model shows consistently better performance.

To illustrate the knowledge learned by the convolutional filters of the DeeReCT-APA model, the convolutional filters of the model are visualized. The aim of visualization is to reveal the important subsequences around polyadenylation sites that activate a specific convolutional filter. In contrast to existing methods, in which the researchers only used sequences in the test set for visualization, the inventors used all sequences in the train and test dataset of F1 for visualization due to the smaller size of the dataset. For visualization, neither the model parameters nor the hyperparameters are tuned on the test set. The learned filters in layer 1 were convolved with all the sequences in the above dataset, and for each sequence, its subsequence (having the same size as the filters) with the highest activation on that filter is extracted and accumulated in a position frequency matrix (PFM). The PFM is then ready for visualization as the knowledge learned by that specific filter. For layer 2 convolutional filters, as they do not convolve with raw sequences during training and testing, directly convolving it with the sequences in the dataset as it was performed for layer 1 would be undesirable. Instead, the layer 2 activations are calculated by a partial forward pass in the network and the subsequences of the input sequences in the receptive field of the maximally-activated neuron is extracted and accumulated in a PFM.

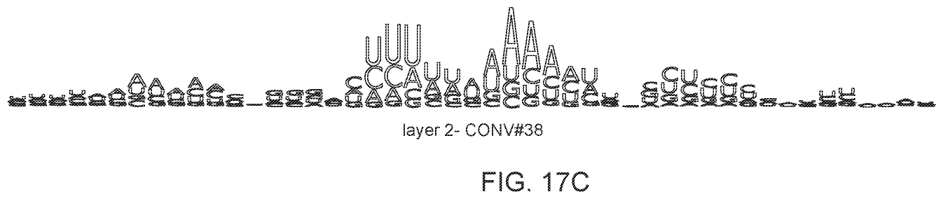

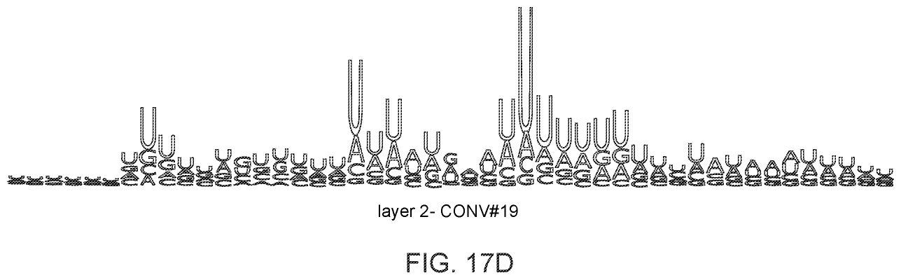

As shown in FIGS. 17A and 17B, the DeeReCT-APA model is able to identify the two strongest PAS hexmer, AUUAAA and AAUAAA [8]. In addition, one of the layer 2 convolutional filters is able to recognize the pattern of a mouse specific PAS hexamer UUUAAA [6], as shown in FIG. 17C. Furthermore, a Poly-U island motif previously reported is also revealed by the DeeReCT-APA model as shown in FIG. 17D.

The above embodiments disclose a novel way to simultaneously predict the usage of all competing PAS within a gene. The novel DeeReCT-APA method incorporates both sequence-specific information through automatic feature extraction by CNN and multiple PAS competition through interaction modeling by the RNN layers. The novel model was trained and evaluated on the genome-wide PAS usage measurement obtained from 3′-mRNA sequencing of fibroblast cells from two mouse strains as well as their F1 hybrid. The DeeReCT-APA model was shown to outperform the state-of-the-art PAS quantification methods on the tasks that they are trained for, including pairwise comparison, highest usage prediction, and ranking task. In addition, it was shown that simultaneously modeling all the PAS of a gene captures the mechanistic competition among the PAS and reveals the genetic variants with regulatory effects on PAS usage.

A method for using a Bi-LSTM layer to model competitive biological processes was proposed recently in [9]. The researchers in [9] used the Bi-LSTM layer to model the usage level of competitive alternative 5′/3′ splice sites. Although the DeeReCT-APA model provides the first-of-its-kind way to model all the PAS of a gene, it still can be improved as all the existing genome-wide PAS quantification datasets used as training data could only sample the limited number of naturally occurring sequence variants. Although in the experiments noted above the two parental strains from which the F1 hybrid mouse was derived are already the evolutionarily most distant ones among all the 17 mouse strains with complete genomic sequences, the number of genetic variants is still rather limited. Thus, it will be desirable to provide a complementary dataset, i.e., to establish a large-scale synthetic APA mini-gene reporter-based system which samples the regulatory effect of millions of random sequences.

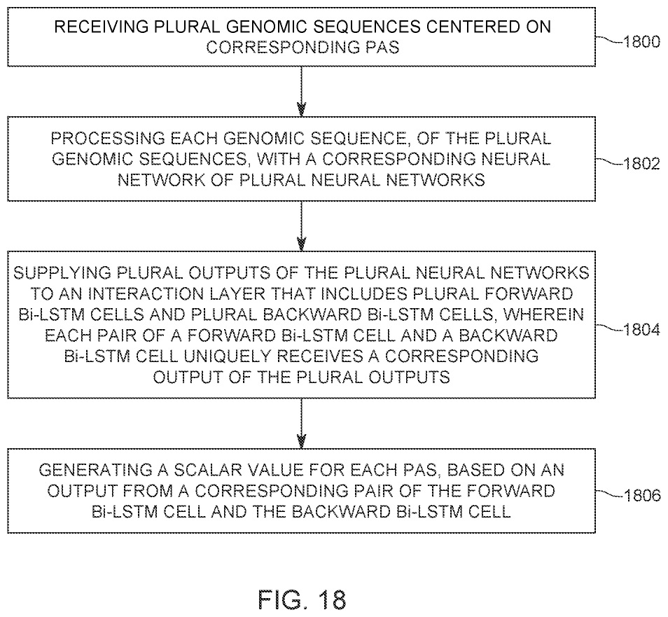

A method for calculating usage of all alternative PAS in a genomic sequence is now discussed with regard to FIG. 18. The method includes a step 1800 of receiving plural genomic sub-sequences 204I centered on corresponding PAS, a step 1802 of processing each genomic sub-sequence 204I, of the plural genomic sequences, with a corresponding neural network 210I of plural neural networks 210I, a step 1804 of supplying plural outputs 206I of the plural neural networks 210I to an interaction layer 220 that includes plural forward Bi-LSTM cells 612I and plural backward Bi-LSTM cells 614I, where each pair of a forward Bi-LSTM cell 612I and a backward Bi-LSTM cell 614I uniquely receives a corresponding output 206I of the plural outputs 206I, and a step 1806 of generating a scalar value for each PAS, based on an output from a corresponding pair of the forward Bi-LSTM cell 612 and the backward Bi-LSTM cell 614.

The method may further include simultaneously considering the plural outputs 206I from the plural neural networks 210I to jointly calculate the plural outputs 206I, where the plural forward Bi-LSTM cells 612I are connected to each other in a given sequence and the plural backward Bi-LSTM cells 614I are connected to each other in a reverse sequence. In one application, each neural network 210I of the plural neural networks 210I includes a convolutional neural network. The convolutional neural network may include a convolution layer, a ReLU layer, and a max-pooling layer. In another application, each of the neural network 210I of the plural neural networks 210I further includes a fully connected layer. In one embodiment, the outputs 206I of the plural neural networks 210I include a sequence motif associated with a corresponding genomic sub-sequence 204I of the plural genomic sub-sequences 204I.

The method may further include a step of generating within the interaction layer 220 a scalar value representing a log-probability 630I for each corresponding PAS, and/or applying a soft-max layer to the scalar values of the PAS to generate a usage percentage value of each PAS, where a sum of all the percentage values is 100%. In one application, the plural forward Bi-LSTM cells 612I and the plural backward Bi-LSTM cells 614I form a recurrent neural network layer, which is configured to capture interdependencies among inputs corresponding to each time step. The recurrent neural network layer has hidden memory cells configured to remember a state for an arbitrary length of time steps, and each time step corresponds to a single PAS.

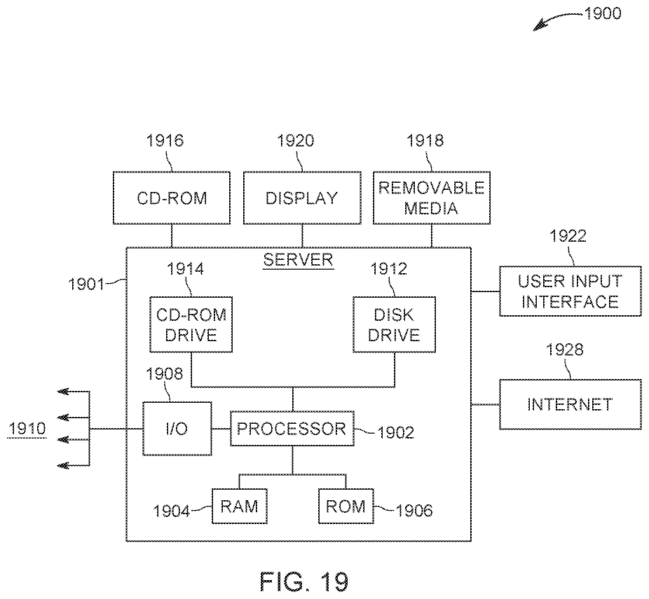

The above-discussed procedures and methods may be implemented in a computing device as illustrated in FIG. 19. Hardware, firmware, software or a combination thereof may be used to perform the various steps and operations described herein. Computing device 1900 of FIG. 19 is an exemplary computing structure that may be used in connection with such a system.

Computing device 1900 suitable for performing the activities described in the embodiments may include a server 1901. Such a server 1901 may include a central processor (CPU) 1902 coupled to a random access memory (RAM) 1904 and to a read-only memory (ROM) 1906. ROM 1906 may also be other types of storage media to store programs, such as programmable ROM (PROM), erasable PROM (EPROM), etc. Processor 1902 may communicate with other internal and external components through input/output (I/O) circuitry 1908 and bussing 1910 to provide control signals and the like. Processor 1902 carries out a variety of functions as are known in the art, as dictated by software and/or firmware instructions.

Server 1901 may also include one or more data storage devices, including hard drives 1912, CD-ROM drives 1914 and other hardware capable of reading and/or storing information, such as DVD, etc. In one embodiment, software for carrying out the above-discussed steps may be stored and distributed on a CD-ROM or DVD 1916, a USB storage device 1918 or other form of media capable of portably storing information. These storage media may be inserted into, and read by, devices such as CD-ROM drive 1914, disk drive 1912, etc. Server 1901 may be coupled to a display 1920, which may be any type of known display or presentation screen, such as LCD, plasma display, cathode ray tube (CRT), etc. A user input interface 1922 is provided, including one or more user interface mechanisms such as a mouse, keyboard, microphone, touchpad, touch screen, voice-recognition system, etc.

Server 1901 may be coupled to other devices, such as sources, sensors, microscopes, etc. The server may be part of a larger network configuration as in a global area network (GAN) such as the Internet 1928, which allows ultimate connection to various landline and/or mobile computing devices.

The disclosed embodiments provide a system, neural network system, and method for Deep Learning based PAS usage prediction in a gene. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Kalkatawi M, Rangkuti F, Schramm M, Jankovic B R, Kamau A, Chowdhary R, et al. Dragon PolyA Spotter: predictor of poly(A) motifs within human genomic DNA sequences. Bioinformatics (Oxford, England) 2012; 28:127-9.
[2] Magana-Mora A, Kalkatawi M, Bajic VB. Omni-PolyA: a method and tool for accurate recognition of Poly(A) signals in human genomic DNA. BMC Genomics 2017; 18:620.
[3] Xie B, Jankovic B R, Bajic V B, Song L, Gao X. Poly(A) motif prediction using spectral latent features from human DNA sequences. Bioinformatics 2013; 29:i316-25.
[4] Leung M K K, Delong A, Frey B J. Inference of the human polyadenylation code. Bioinformatics 2018; 34:2889-98.
[5] Arefeen A, Jiang T, Xiao X. DeepPASTA: Deep neural network based polyadenylation site analysis, 2019.
[6] Xia Z, Li Y, Zhang B, Li Z, Hu Y, Chen W, et al. DeeReCT-PolyA: a robust and generic deep learning method for PAS identification. Bioinformatics 2018: bty991-bty.
[7] Hochreiter S, Schmidhuber J. Long Short-Term Memory. Neural Computation 1997; 9:1735-80.
[8] Xiao M S, Zhang B, Li Y S, Gao Q, Sun W, Chen W. Global analysis of regulatory divergence in the evolution of mouse alternative polyadenylation. Molecular Systems Biology 2016; 12:890.
[9] Zuberi K, Gandhi S, Bretschneider H, Frey B J, Deshwar A G. COSSMO: predicting competitive alternative splice site selection using deep learning. Bioinformatics 2018; 34:i429-i37.

1. A method for calculating usage of all alternative polyadenylation sites in a genomic sequence, the method comprising:
receiving plural genomic sub-sequences centered on corresponding PAS;
processing each genomic sub-sequence of the plural genomic sequences, with a corresponding neural network of plural neural networks;
supplying plural outputs of the plural neural networks to an interaction layer that includes plural forward Bidirectional Long Short Term Memory Network (Bi-LSTM) cells and plural backward Bi-LSTM cells, wherein each pair of a forward Bi-LSTM cell and a backward Bi-LSTM cell uniquely receives a corresponding output, of the plural outputs, from a corresponding neural network; and
generating a scalar value for each PAS, based on an output from a corresponding pair of the forward Bi-LSTM cell and the backward Bi-LSTM cell.

2. The method of claim 1, further comprising:
simultaneously considering the plural outputs from the plural neural networks to jointly calculate the scalar values for all the PAS.

3. The method of claim 1, wherein the plural forward Bi-LSTM cells are connected to each other in a given sequence and the plural backward Bi-LSTM cells are connected to each other in a reverse sequence.

4. The method of claim 1, wherein each neural network of the plural neural networks includes a convolutional neural network.

5. The method of claim 4, wherein the convolutional neural network includes a convolution layer, a ReLU layer, and a max-pooling layer.

6. The method of claim 4, wherein each of the neural network of the plural neural networks further includes a fully connected layer.

7. The method of claim 1, wherein the outputs of the plural neural networks include a sequence motif associated with a corresponding genomic sub-sequence of the plural genomic sub-sequences.

8. The method of claim 1, further comprising:
generating within the interaction layer a scalar value representing a log-probability for each corresponding PAS.

9. The method of claim 8, further comprising:
applying a soft-max layer to the scalar values of the PAS to generate a usage percentage value of each PAS, where a sum of all the percentage values is 100%.

10. The method of claim 1, wherein the plural forward Bi-LSTM cells and the plural backward Bi-LSTM cells form a recurrent neural network layer, which is configured to capture interdependencies among inputs corresponding to each time step.

11. The method of claim 10, wherein the recurrent neural network layer has hidden memory cells configured to remember a state for an arbitrary length of time steps, and each time step corresponds to a single PAS.

12. A computing device for calculating usage of all alternative polyadenylation sites (PAS) in a genomic sequence, the computing device comprising:
an interface configured to receive plural genomic sub-sequences centered on corresponding PAS; and
a processor connected to the interface and configured to,
process each genomic sub-sequence of the plural genomic sequences, with a corresponding neural network of plural neural networks;
supply plural outputs of the plural neural networks to an interaction layer that includes plural forward Bidirectional Long Short Term Memory Network (Bi-LSTM) cells and plural backward Bi-LSTM cells, wherein each pair of a forward Bi-LSTM cell and a backward Bi-LSTM cell uniquely receives a corresponding output of the plural outputs; and
generate a scalar value for each PAS, based on an output from a corresponding pair of the forward Bi-LSTM cell and the backward Bi-LSTM cell.

13. The computing device of claim 12 wherein the processor is further configured to:
simultaneously consider the plural outputs from the plural neural networks to jointly calculate the scalar values for all the PAS.

14. The computing device of claim 12, wherein the plural forward Bi-LSTM cells are connected to each other in a given sequence and the plural backward Bi-LSTM cells are connected to each other in a reverse sequence.

15. The computing device of claim 12, wherein the plural forward Bi-LSTM cells and the plural backward Bi-LSTM cells form a recurrent neural network layer, which is configured to capture interdependencies among inputs corresponding to each time step.

16. The computing device of claim 15, wherein the recurrent neural network layer has hidden memory cells configured to remember a state for an arbitrary length of time steps, and each time step corresponds to a single PAS.

17. A neural network system for calculating usage of all alternative polyadenylation sites (PAS) in a genomic sequence, the system comprising:
plural neural networks configured to receive plural genomic sub-sequences centered on corresponding PAS, wherein the plural neural networks are configured to process the genomic sub-sequences such that each neural network processes only a corresponding genomic sub-sequence of the genomic sequence;
an interaction layer configured to receive plural outputs of the plural neural networks, wherein the interaction layer includes plural forward Bidirectional Long Short Term Memory Network (Bi-LSTM) cells and plural backward Bi-LSTM cells, and wherein each pair of a forward Bi-LSTM cell and a backward Bi-LSTM cell uniquely receives a corresponding output of the plural outputs; and
an output layer configured to generate a scalar value for each PAS, based on an output from a corresponding pair of the forward Bi-LSTM cell and the backward Bi-LSTM cell.

18. The neural network system of claim 17, wherein the plural neural networks are further configured to:
simultaneously consider the plural outputs from the plural neural networks to jointly calculate the scalar values of all the PAS.

19. The neural network system of claim 17, wherein the plural forward Bi-LSTM cells are connected to each other in a given sequence and the plural backward Bi-LSTM cells are connected to each other in a reverse sequence.

20. The neural network system of claim 17, wherein the plural forward Bi-LSTM cells and the plural backward Bi-LSTM cells form a recurrent neural network layer, which is configured to capture interdependencies among inputs corresponding to each time step, and wherein the recurrent neural network layer has hidden memory cells configured to remember a state for an arbitrary length of time steps, and each time step corresponds to a single PAS.
